(19) United States
(12) Patent Application Publication
(10) Pub. No.: US 2024/0207241 A1
(43) Pub. Date: Jun. 27, 2024

(54) Method for Promoting Functional Cure of AIDS by Reducing HIV Reservoir with Lenalidomide

(71) Applicant: The First Affiliated Hospital Zhejiang University School of Medicine, Hangzhou (CN)

(72) Inventors: Biao Zhu, Hangzhou (CN); Xiang Liu, Hangzhou (CN); Xueling Zhu, Hangzhou (CN); Yongzheng Guo, Hangzhou (CN); Xiaorong Peng, Hangzhou (CN); Qing Cao, Hangzhou (CN)

(21) Appl. No.: 18/391,649

(22) Filed: Dec. 20, 2023

Related U.S. Application Data
(60) Provisional application No. 63/434,103, filed on Dec. 21, 2022.

(51) Int. Cl. A61K 31/454; A61P 31/18

(52) U.S. Cl. CPC A61K 31/454; A61P 31/18

(57) ABSTRACT

The present invention provides a method for promoting functional cure of AIDS by reducing HIV reservoir with lenalidomide, the method including administering a therapeutically effective amount of lenalidomide to a patient. Also provided is a method for reducing intracellular P24 and cell-related HIV RNA in PBMCs derived from HIV patients in vitro with lenalidomide, including orally administering lenalidomide at 25 mg/d at days 1 to 21 in a 28-day cycle, and continuing ART for 48 weeks. Further provided is a method for inhibiting latent activator-induced activation of latent HIV cell line U1 with lenalidomide, including orally administering lenalidomide at 25 mg/d at days 1 to 21 in a 28-day cycle, and continuing ART for 48 weeks.

### CROSS-REFERENCE TO RELATED APPLICATIONS

The present application claims priority from U.S. Provisional Patent Application No. 63/434,103 filed on Dec. 21, 2022, the contents of which are incorporated herein by reference in their entirety.

### TECHNICAL FIELD

The present invention relates to the field of functional cure of HIV, and particularly to a method for promoting functional cure of AIDS by reducing HIV reservoir with lenalidomide.

### BACKGROUND

The persistence of latent cells in HIV reservoir is a major barrier to curative strategies. To clear the HIV reservoir, the researchers propose a “shock and kill” strategy, aiming at re-activating latent cells by latent reversal agents (LRAs) of small molecular compounds and killing the re-activated cells by the cytotoxic effects or the host's immune system. Unfortunately, the “shock and kill” strategy not only faces challenges in re-activating most latent proviruses in the body, but also has difficulties in inducing HIV-1-specific immune-mediated clearance. So far, there is no research on LRA that is confirmed to actually reduce the latent reservoir in patients. Later, the researchers focus on another functional cure method, that is, the “block and lock” strategy. This treatment aims to stably inhibit the transcription of viral reservoir with a latency-promoting agent (LPA), and lock latent HIV cells in deep latency, so that all proviruses can be silenced permanently even after the treatment is stopped. LPA is also used to reduce the residual viremia during treatment, and prevent the virus from recurrence after the treatment is stopped. Many cell regulatory proteins or factors related to HIV transcription and silencing are potential targets of LPA. In literatures involving the “block and lock” strategy, Valente Lab reported that dihydrocortistatin A (dCA), a TAT-mediated HIV-1 transcription inhibitor, can enhance the epigenetic silencing of HIV-1 promoter, causing the long-term inactivation of proviruses to inhibit the replication of the virus. It is generally accepted as the most effective LPA at present. In addition, small molecules acting on histone and DNA methylation, curaxin 100 targeting facilitates chromatin transcription (FACT) complex, compounds blocking mTOR and small molecule integrase inhibitor LEDGINs targeting LEDGF/p75 are all potential LPA inhibiting latent cell replication. However, most studies on LPA are in vitro, and no clinical cohort study has been reported.

Our research focuses on the effect of immunomodulators on inhibiting the replication of latent HIV cells. HIV infection will lead to elevated inflammation level in the body, the replication of latent viral reservoir will aggravate the inflammation, and the elevated inflammation level will stimulate the transcription of latent HIV cells, thus forming a vicious circle. Inflammatory cytokines are involved in the maintenance and transcription of HIV reservoir. Early studies have shown that γ-chain cytokines (IL-2, IL-7, IL-15) are the key driving force responsible for the steady proliferation of HIV reservoir, and promote the viral transcription of latent cells. IL-2 is also the first LRA that is used in clinic to activate the latent cells, but it is not used anymore because of extensive T cell activation. TNF-a, as a positive control agent in various cell experiments of HIV reservoir, is confirmed to have ability to activate the replication of latent cells. In addition, it participates in the HIV transcription through various inflammatory pathways (JAK-STAT, MAPK, PI3K-Akt, NF-κB) in the body. Potential immunomodulators are chosen to inhibit the release of proinflammatory cytokines, block the targets of inflammatory pathways, thus reducing the level of inflammation, inhibiting the replication of latent HIV cells and silencing the HIV reservoir in the body.

Lenalidomide is the initially available immunomodulator. As an immunomodulator, it is widely used in the treatment of diseases such as hematological tumors, multiple myeloma, lymphoma and MDS, with verified safety. The mechanism of action is not completely clear. However, many studies have shown that lenalidomide, an immunomodulator, can inhibit the production of pro-inflammatory cytokines TNF-α, IL-1, IL-6, and IL-12, and increase the production of anti-inflammatory cytokine IL-10 in human PBMC. However, it has also been reported that lenalidomide can regulate T cell immunity, activate T cell function, and increase the secretion of cytokines such as IL-2, TNF-α and IFN-γ. It is assumed that lenalidomide has dual immunomodulatory effects, and has different effects in different diseases. At present, there is no research on the effect of lenalidomide on HIV reservoir. Herein, lenalidomide is used to act on latent HIV cells and primary cells and HIV-infected patients are recruited for clinical cohort study, to confirm that lenalidomide has effect on latent HIV reservoir.

### SUMMARY

In view of the above technical problems, the present invention mainly aims at inhibiting the replication of latent HIV reservoir, reducing the chronic inflammation in HIV patients, and effectively solving the problem of functional cure of HIV. The method includes reducing inflammatory cytokines and the index of latent HIV reservoir in peripheral blood and central nervous system of HIV patients with lenalidomide, and inhibiting the reverse transcription of HIV viruses by the in-vitro effect of lenalidomide on latent HIV cell line U1.

A method for promoting functional cure of AIDS by reducing HIV reservoir with lenalidomide in a patient includes administering a therapeutically effective amount of lenalidomide to the patient.

HIV patients with persistent central inflammation after screening of HIV-related cryptococcal meningitis (CM) are orally administered with lenalidomide for 48 weeks, then inflammatory cytokines in peripheral blood and cerebrospinal fluid (CSF) of the patients are detected, PBMCs are extracted, to detect the intracellular HIV RNA level of the patients.

Lenalidomide is orally administered at 25 mg at days 1 to 21 in a 28-day cycle, and ART is continued for 48 weeks.

The HIV patients are HIV patients with persistent central inflammation after screening of HIV-related CM.

The patients still have central damage after receiving standard anti-fungal therapy, elevated cerebrospinal fluid (CSF) protein level, and plasma HIV RNA of <50 copies/ml and CD4 T cell count of >100 cells/mm3 after receiving ART for at least 48 weeks.

After administration, inflammatory cytokines in the peripheral blood and CSF of the patients are detected, and PBMCs are extracted, to detect the intracellular HIV RNA level of the patients.

Lenalidomide acts on PBMCs derived from HIV patients in vitro, and the changes of inflammatory cytokines and the intracellular P24 and HIV RNA levels are detected.

The latent HIV cell line U1 is pretreated with lenalidomide, a latent activator (TNF-a, PMA, or LPS) is added, and the P24 content in the supernatant of U1 cell culture and the HIV RNA level in the cytoplasm are detected.

The present invention has the following beneficial effects: A method for promoting functional cure of AIDS by reducing HIV reservoir with lenalidomide is provided. The persistence of latent cells in HIV reservoir is a major barrier to curative strategies. Chronic immune activation promotes the occurrence of various non-AIDS events in AIDS patients and participates in the maintenance and activation of latent HIV reservoir. Lenalidomide is found to reduce the inflammatory cytokines IL-6 and TNF-a and the inflammatory markers CRP and D-dimer in peripheral blood of HIV patients, and thus reduce the HIV RNA level related to the indicator cells in the latent HIV reservoir through the reduction of cytokines in peripheral blood. It is also confirmed in the primary HIV cell line and latent cell line U1 in vitro that lenalidomide reverses the activation of U1 cells by a latent activator (TNF-a, PMA, or LPS), reduces the release of HIV p24 protein by the cells and the content of HIV RNA in the cytoplasm. The present invention is used for the functional cure of HIV patients, expands the new use of lenalidomide, and provides a new approach to the functional cure of HIV.

### BRIEF DESCRIPTION OF THE DRAWINGS

FIG. 1 shows dynamic changes of inflammatory markers and cytokines in peripheral blood of cohort patients. Longitudinal dynamic changes of (A) TNF-α, (B) IL-6, (C) CRP and (D) D-dimer during 48 weeks of treatment with lenalidomide are shown. The P value representing the difference between various time points is shown on the right side of the panel. The dynamic follow-up data of the subjects are expressed in different colors.

FIG. 2 shows dynamic changes of virological indicators of cohort patients. Longitudinal dynamic changes of (A) HIV-1 RNA, (B) CA HIV-1 RNA, (C) CA HIV-1 RNA/total HIV-1 DNA, and (D) total HIV-1 DNA during 48 weeks of treatment with lenalidomide are shown. The P value representing the difference between various time points is shown on the right side of the panel. The dynamic follow-up data of the subjects are expressed in different colors.

FIG. 3 shows effects of lenalidomide (0.5, 1, 2, 5, 10, 20 μmol/L) on the survival rate of U1 cells after 24 hrs of action (A); quantitative detection of P24 by ELISA in the supernatant obtained after U1 cells are pretreated with different concentrations of lenalidomide at 1, 2, and 5 μmol/L, and then the U1 cells are stimulated with PMA (20 nmol/L) for 24 hrs (B); quantitative detection of P24 by ELISA in the supernatant obtained after U1 cells are pretreated with different concentrations of lenalidomide at 1, 2, and 5 μmol/L, and then the U1 cells are stimulated with TNF-a at 20 μg/ml for 24 hrs (C); and quantitative detection of P24 by ELISA in the supernatant obtained after U1 cells are pretreated with different concentrations of lenalidomide at 1, 2, and 5 μmol/L, and then the U1 cells are stimulated with LPS at 10 μg/ml for 24 hrs (D). * represents P<0.05, ** represents P<0.01, *** represents P<0.001, and ns means no correlation.

FIG. 4 shows the inhibition of lenalidomide on the expression of viral mRNA level in latent cells. The mRNA expressions of Gag, Rev, multi-spliced RNA, single-spliced RNA and non-spliced RNA in negative control group (red), positive control group (purple) and experimental group (green) are indicated with different colors. * represents P<0.05, ** represents P<0.01, and *** represents P<0.001.

FIG. 5 shows that lenalidomide inhibits the reactivation of virus in HIV-1 primary cell CD8-PBMCs. A compares intracellular expressions of HIV-1 core antigen P24 in three groups of CD8-PBMCs by flow cytometry. B is a statistical graph of intracellular expressions of HIV-1 core antigen P24 in three groups of CD8-PBMCs derived from 5 patients; C shows the CA HIV RNA levels in CD8-PBMCs derived from 5 patients by RT-PCR. * represents p<0.05, and ** represents p<0.01.

FIG. 6 shows that lenalidomide regulates the secretion of cytokines in PBMCs in vitro. A shows the detection by flow cytometry of cytokines in two groups of PBMCs treated with lenalidomide. B is a statistic graph showing the effects of lenalidomide on cytokines TNF-α, IL-6 and IL-10 in two groups having three replicates. * represents p<0.05, and ** represents p<0.01.

FIG. 7 shows the effects of the supernatant of PBMCs from HIV-1 patients treated with lenalidomide in vitro on the virus activation of latent cell lines J-lat10.6 and C11 cells. A shows the activation of J-lat10.6 cells by the supernatant in the experimental group and the control group. B is a statistic graph showing the different activations of J-lat10.6 cells by two groups of supernatants having three replicates. C shows the activation of C11 cells by the supernatant in the experimental group and the control group. D is a statistic graph showing the different activations of C11 cells by two groups of supernatants having three replicates. *** represents P<0.001, and **** represents P<0.0001.

### DETAILED DESCRIPTION

Unless otherwise specified, the reagents used in this application are all commercially available, and lenalidomide is purchased from Qilu Pharmaceutical Co., Ltd. (Qipuyi® lenalidomide).

### Example 1. Lenalidomide Reduces Chronic Inflammation and Cell-Related HIV RNA Level in HIV Patients

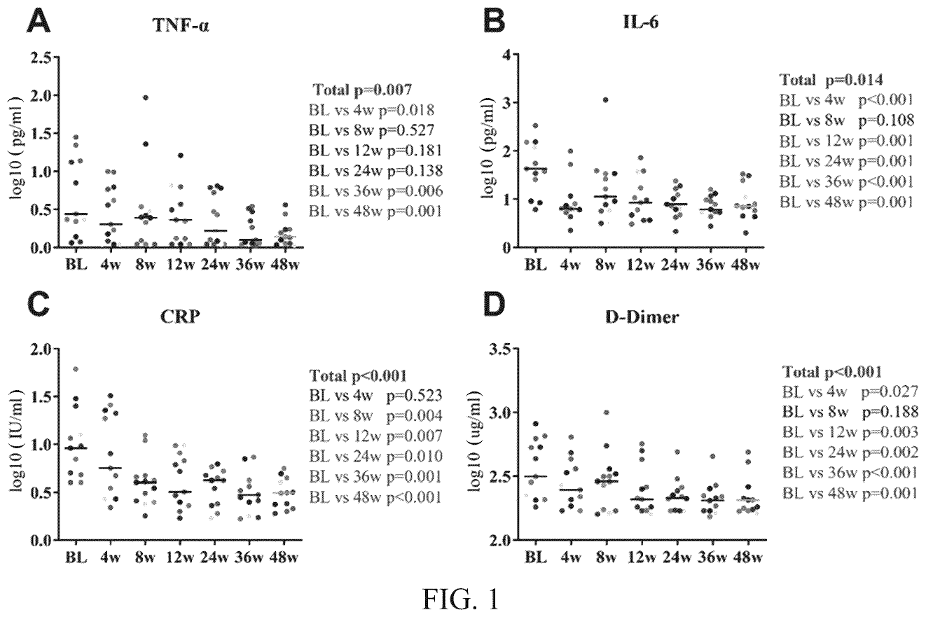

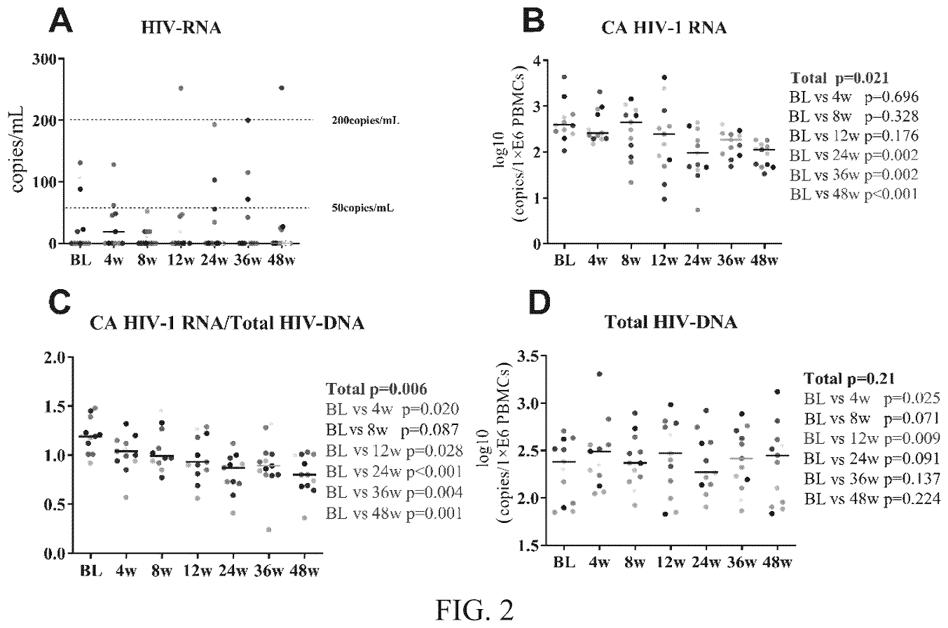

HIV patients with CM after antiretroviral therapy (ART) were recruited. These participants had central damage after receiving standard anti-fungal therapy, elevated cerebrospinal fluid (CSF) protein level, age of 18 years or more, and plasma HIV RNA of <50 copies/ml and CD4 T cell count of >100 cells/mm3 after receiving ART for at least 48 weeks. Lenalidomide (25 mg) was orally administered at days 1 to 21 in a 28-day cycle, and ART was continued for 48 weeks. The participants were followed up at 7 points (base line 0w, 4w, 8w, 12w, 24w, 36w, and 48w). Inflammatory cytokines in CSF and peripheral blood were detected at each follow-up point, and PBMCs were extracted from the patients and detected for indicators of HIV reservoir. After lenalidomide is used, CRP and D-dimer of HIV subjects decrease after 7 weeks and 12 weeks of lenalidomide treatment respectively, and cytokines IL-6 and TNF-a decrease significantly after lenalidomide treatment. Lenalidomide significantly reduces the levels of TNF-a and IL-6 in CSF. For CA HIV-RNA, lenalidomide has a great influence. From the decline trend of 13 patients, it can be seen that CA RNA declines obviously from the 24th week after lenalidomide treatment, and the decline magnitude is even more obvious at the 48th week. The subjects Nos. 110 and 113 have a decline that exceeds 2 log order of magnitude. After statistical analysis, it can be found that there are significant differences between the baseline level and the levels at the 24th 36th, and 48th weeks (FIGS. 1-2).

### Example 2. Lenalidomide Inhibits HIV Reactivation in Latent Cell Lines In Vitro

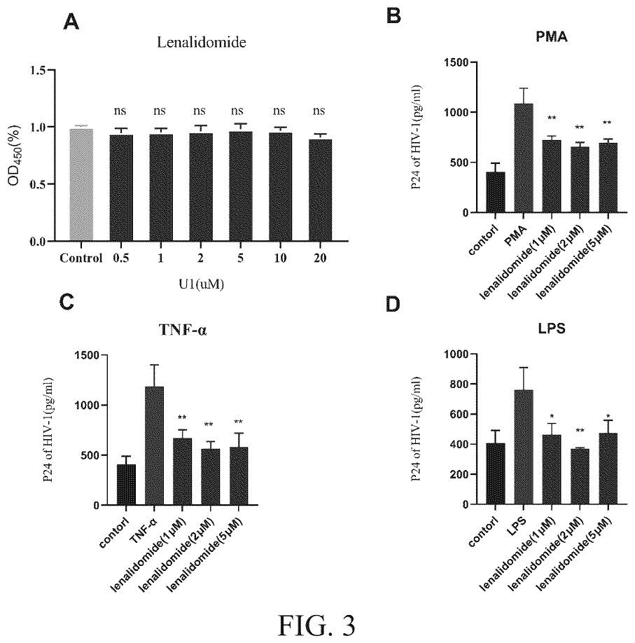

CCK8 was used to evaluate the safety of lenalidomide when U1 cells were treated with it in vitro. After U1 cells were treated with various concentrations of lenalidomide (0.5, 1, 2, 5, 10, and 20 μmol/L) for 24 hrs, the survival rate of U1 cells was detected by CCK8 kit. The results show that after U1 cells are treated with the various concentrations of lenalidomide, the absorbance does not change significantly. Through calculation, it is found that the survival rate of U1 cells is less affected by lenalidomide (A in FIG. 3). There is no significant difference between the control group and various concentration groups and between the various concentration groups. Lenalidomide has a high safety when acting on latent cell lines in vitro.

U1 cells were pretreated with various concentrations of lenalidomide (1, 2, and 5 μmol/L) for 24 hrs, and then the U1 cells pre-treated with lenalidomide were stimulated with a latent activator (TNF-a, PMA, or LPS). The U1 cells in the negative control group were cultured alone, and the U1 cells in the positive control group were not pretreated with lenalidomide. The activation of P24 protein in the supernatant of latent cells U1 was detected by ELISA. (1) After the U1 cells are stimulated with PMA (20 nmol/L) for 24 hrs, compared with the negative control group, P24 in the experimental group and the positive control group increase significantly, and the difference is statistically significant. Compared with the positive control group, the P24 contents decline in the supernatants of the three experimental groups pretreated with 1, 2, and 5 μmol/L lenalidomide, and the difference is statistically significant (P=0.0029, 0.0019, and 0.0017, B in FIG. 3). The difference between various concentrations of lenalidomide groups is not obvious. (2) In the experiment where TNF-α of 20 μg/ml is used as the latent activator, similar results are observed. Compared with the negative control group, P24 in the experimental group and the positive control group increase significantly, and The P24 contents in the supernatant of the three experimental groups are significantly lower than that of the positive control group, and there are statistical difference therebetween (P=0.0079, 0.0026, and 0.0029, C in FIG. 3), There is no obvious difference observed in the inhibition between the three groups. (3) The study of endotoxin (LPS, 10 μg/ml) as a latent activator is rare. It is found that LPS can activate the latent cell line U1, and its activation effect can be inhibited by lenalidomide. The P24 contents in the supernatant of the three experimental groups are significantly lower than that of the positive control group, and there are statistical difference therebetween (P=0.0171, 0.0034, and 0.0204, D in FIG. 3). There is no significant difference between the experimental groups, and lenalidomide does not show concentration-dependent inhibitory effect.

### Example 3. Lenalidomide can Inhibit the Expression of HIV-RNA mRNA Associated with U1 Cells

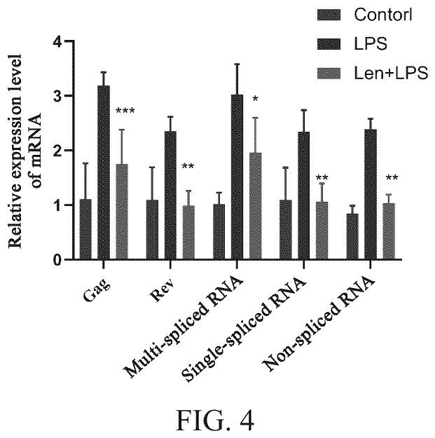

After the HIV-1 latent cell line is activated by activator, the expression of viral mRNA in the cells is increased, causing the production of virions. After the U1 cells are pre-treated with lenalidomide and then the U1 cells are activated with a latent activator, it is found that lenalidomide can inhibit the expression of CA HIV RNA mRNA (FIG. 4). The U1 cells were divided into three groups. The negative control group was added with the same amount of culture medium. In the positive control group, cells were pretreated with the culture medium, and LPS was added as an activator after 24 hrs. In the experimental group, lenalidomide (2 μmol/L) was added to pretreat the cells for 24 hrs and then LPS was added to activate the cells. (1) After the latent activator LPS of 10 μg/ml is used for 24 hrs, the mRNA levels of Gag and Rev protein in U1 cells are significantly increased compared with those in the negative control group, and the difference is statistically significant. The mRNA levels of Gag and Rev protein in U1 cells pretreated with lenalidomide are basically inhibited, significantly lower than that in the positive control group, and basically the same as that in the negative control group (P=0.0007, 0.0012). (2) The expression levels of multi-spliced RNA, single-spliced RNA and non-spliced RNA in U1 were also detected. Similar to viral protein mRNA, lenalidomide significantly inhibits the transcription levels of different transcripts in U1 cells, with statistical significance (P=0.0103, 0.0022, and 0.0015).

### Example 4

### Lenalidomide Inhibits the Reactivation of Virus in HIV-1 Primary Cell Line (CD8-PBMCs)

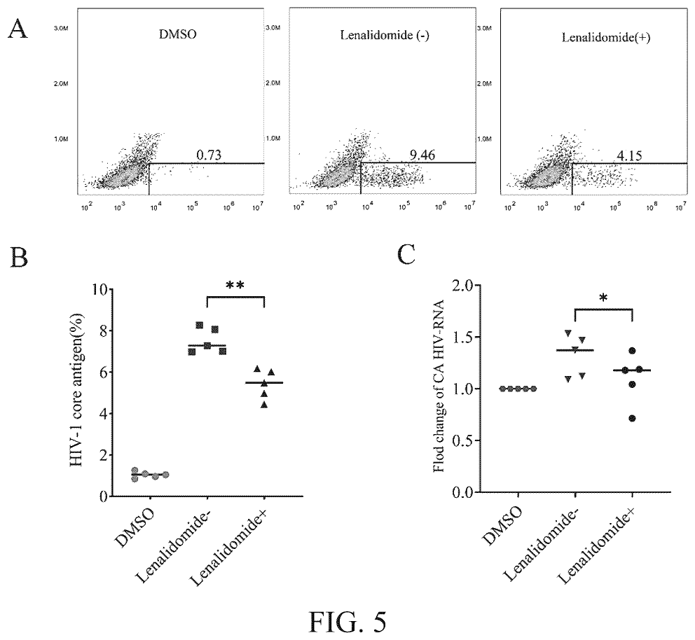

Lenalidomide is confirmed to inhibit the transcription of virus in HIV-1 latent cell line, and whether it has the same effect in HIV primary cells is unknown. CD8-PBMCs were divided into an experimental group and a positive control group and treated with or without lenalidomide for 5 days. Both groups were supplemented with LPS irregularly. In the negative control group, the same amount of DMSO was added for control culture throughout the whole process. After 5 days, the cells were broken, and the difference in expression of intracellular P24 protein in CD8-PBMCs was detected by flow cytometry. As shown in A in FIG. 5, in the positive control group, the intracellular HIV-1 core P24 in CD8-PBMCs reaches 9.76% after the continuous action of activator. However, after lenalidomide treatment, the expression of intracellular antigen P24 in the experimental group decreases to 4.15%, and it is only 0.73% in the DMSO control group. After repeated tests of five samples lenalidomide is found to significantly reduce the percentage of HIV-1 core P24 in primary CD8-PBMCs (P=0.0011, B in FIG. 5). The differences in the expression of CA HIV RNA in the five samples were also detected. It is found that compared with the positive control group, the average amount of CA HIV-1 RNA in the lenalidomide treatment group is decreased by 0.49 time, which is statistically significant (P=0.0018, C in FIG. 5). The data of primary cell latent model shows that lenalidomide is an effective inhibitor of reactivation of viruses in HIV-1 latent cell (FIG. 5).

### Example 5 Lenalidomide Regulates the Levels of TNF-α, IL-6 and IL-10 in Supernatant of PBMCs Derived from HIV-1 Patients to Inhibit HIV Replication

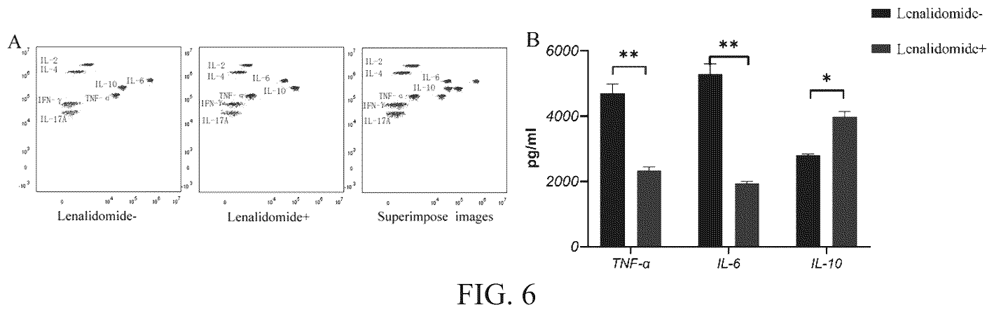

Lenalidomide is confirmed to effectively inhibit the secretion of cytokines in HIV-1 patients. The in-vitro regulatory effect of lenalidomide was verified as follows. PBMCs were separated from peripheral blood donated by HIV-1 patients, and divided into a control group and an experimental group. The control group was cultured alone, and the experimental group was co-cultured with lenalidomide. After 24 hrs, the supernatant was taken for cytokine detection. The level of cytokines in the supernatant was measured by a cytometric bead array kit. Compared with the control group (without lenalidomide), TNF-α and IL-6 in the supernatant of the experimental group decrease significantly (P=0.004, P<0.0001), but IL-10 increases significantly (P<0.0001) (FIG. 6). Lenalidomide not only inhibits the production of pro-inflammatory cytokines TNF-α and IL-6 in vitro, but also promotes the secretion of IL-10.

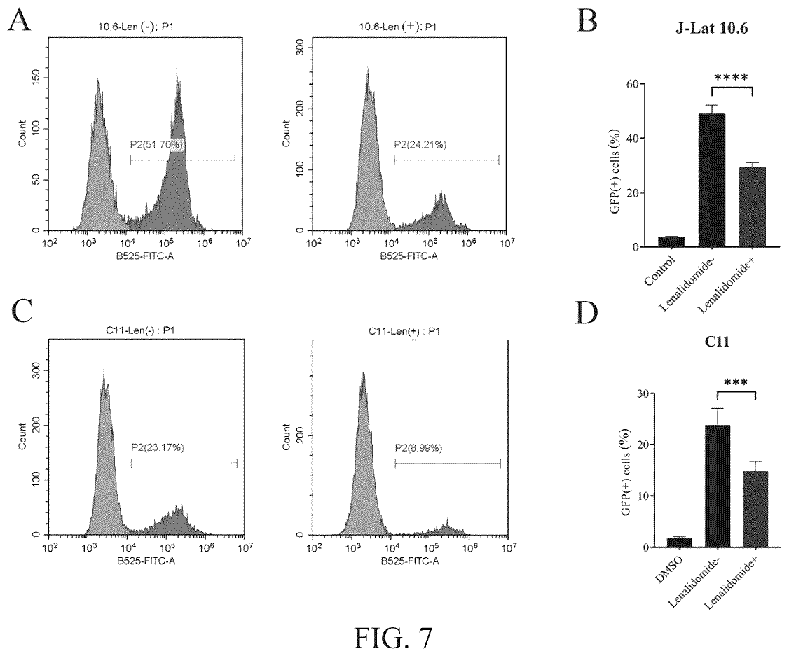

Lenalidomide has a powerful immunomodulatory effect, and can inhibit the release of pro-inflammatory cytokines in PBMCs derived from HIV patients in vitro and promote the secretion of anti-inflammatory cytokine IL-10. However, whether this degree of immunomodulation can inhibit HIV replication was not confirmed. The latent cell lines J-lat10.6 and C11 cells were used for the experiment. The supernatants of the experimental group and control group were used to stimulate J-lat10.6 and C11 cells, respectively. (1) In the experiment with J-lat 10.6 cells, it is found that the supernatant of the control group activates GFP by 51.7%, and the supernatant added with lenalidomide inhibits J-lat10.6 GFP by 24.21% (A-B in FIG. 7). (2) From the experiment where C11 cells are treated with the supernatant of the two groups, it can be seen that the lenalidomide group also reduces the GFP activation of C11 cells from 23.17% to 8.09%, and the difference is statistically significant (C-D in FIG. 7). Lenalidomide can regulate the release of inflammatory cytokines in PBMCs from HIV-1 patients in vitro, and this bypass pathway can inhibit virus reactivation in HIV-1 latent cell lines.

What is claimed is:

1. A method for promoting functional cure of AIDS by reducing HIV reservoir with lenalidomide in a patient, comprising administering a therapeutically effective amount of lenalidomide to the patient.

2. The method according to claim 1, wherein lenalidomide is orally administered at 25 mg/d at days 1 to 21 in a 28-day cycle, and ART is continued for 48 weeks.

3. The method according to claim 1, wherein lenalidomide is used to reduce Th1 inflammatory cytokines in peripheral blood and cerebrospinal fluid of HIV patients.

4. The method according to claim 1, wherein lenalidomide is used to relieve chronic inflammatory state in HIV patients.

5. The method according to claim 1, wherein lenalidomide is used to reduce cell-related HIV RNA level in HIV patients.

6. The method according to claim 5, wherein lenalidomide is used to reduce intracellular P24 and cell-related HIV RNA in PBMCs derived from HIV patients in vitro.

7. The method according to claim 1, wherein the patients are HIV patients with persistent central inflammation after screening of HIV-related cryptococcal meningitis.

8. The method according to claim 1, wherein the patients still have central damage after receiving standard anti-fungal therapy, elevated cerebrospinal fluid (CSF) protein level, and plasma HIV RNA of <50 copies/ml and CD4 T cell count of >100 cells/mm3 after receiving ART for at least 48 weeks.

9. The method according to claim 8, wherein after lenalidomide administration, inflammatory cytokines in the peripheral blood and CSF of the patients are detected, and PBMCs are extracted to detect the intracellular HIV RNA level of the patients.

10. A method for reducing intracellular P24 and cell-related HIV RNA in PBMCs derived from HIV patients in vitro with lenalidomide, comprising orally administering lenalidomide at 25 mg/d at days 1 to 21 in a 28-day cycle, and continuing ART for 48 weeks.

11. A method for inhibiting latent activator-induced activation of latent HIV cell line U1 with lenalidomide, comprising orally administering lenalidomide at 25 mg/d at days 1 to 21 in a 28-day cycle, and continuing ART for 48 weeks.

12. The method according to claim 11, wherein the latent activator is TNF-a, PMA or LPS.
